(19) United States
(12) Patent Application Publication
(10) Pub. No.: US 2010/0161068 A1
(43) Pub. Date: Jun. 24, 2010

(54) PROSTHESIS FOR PART OF A JOINT, WITH AN EXPANDABLE SHAFT

(75) Inventors: Nicola Lindner, Wurmlingen (DE); Cliff-Georg Reitzig, Rietheim-Weilheim (DE); Stephan Eckhof, Rietheim-Weilheim (DE); Thomas Feldhaus, Tuttlingen (DE)

Correspondence Address: COLLARD & ROE, P.C., 1077 NORTHERN BOULEVARD, ROSLYN, NY, 11576, US

(73) Assignee: ZRINSKI AG

(21) Appl. No.: 12/452,560

(22) PCT Filed: May 9, 2008
(86) PCT No.: PCT/IB2008/001198

§ 371 Date: Jan. 8, 2010

(30) Foreign Application Priority Data
Jul. 9, 2007 (DE) 20 2007 009 619.4

(51) Int. Cl. A61F 2/42

(52) U.S. Cl. 623/21.15

(57) ABSTRACT

The invention relates to a joint part prosthesis, particularly for a finger joint, consisting either of a proximal or a distal component as well as a shaft extending away from this component, to be mounted in a bone. The invention provides that the shaft (4) is at least partially formed as an expansion sleeve (7).

### TECHNICAL FIELD

The invention relates to a finger joint part prosthesis, particularly for a finger joint. It consists of either a proximal or a distal component, as well as a shaft that extends away from this component, to be mounted in a bone.

### STATE OF THE ART

Artificial finger joints consist essentially of two elements, namely a proximal and a distal component. A component has a convex joint head that interacts with the other part, which demonstrates a convex joint socket.

Artificial finger joints are inserted between the metacarpal bone and the finger bone, or between individual finger bones. It is also known that exclusively the functionally defective finger joint can be replaced, so that only one artificial component is present.

Such joints have to be inserted if degenerative joint diseases such as osteoarthritis, post-traumatic arthritis, or rheumatoid arthritis of the joints in question are present. Another alternative that retains mobility of the individual finger members is a total joint replacement.

From the state of the art, particularly from EP 1203569 A (FINSBURY (DEVELOPMENT) LIMITED LEATHERHEAD) Nov. 3, 2000, finger joint implants are known in the configuration of so-called PIP shaft implants. They have a proximal or a distal component, which components interact accordingly, each having a shaft that points away from these components. The shaft is generally configured to be conical and is introduced into the bone marrow of a bone. In order to allow introduction, the interior of the bone is partly cleared out using a clearing tool, and the implant is driven into the bone using a hammer-like instrument. Fixation takes place in such a manner that the diameter of the cleared bore is smaller than the outside diameter of the shaft, so that a kind of press fit is formed between the shaft and the interior of the bone.

The implant itself consists of a material having a modulus of elasticity similar to bone. This modulus of elasticity similar to bone avoids so-called stress shielding and thus promotes bone build-up. Additionally, fixation of the implant in the medullary space of the bone is improved in this manner.

### Disadvantages of the State of the Art

Products according to the state of the art have disadvantages such as implant loosening due to insufficient fixation and insufficient connection with the bone, as well as high wear values, thereby causing corresponding friction wear. The shaft-like construction within the medullary space creates sufficient room for itself if loosened, so that an implant that was fixed in place previously is no longer functionally correct. The consequences of this are pain caused by migration or even fractures.

### Task of the Invention

It is therefore the task of the invention to further develop a joint part prosthesis, particularly for finger joints, in such a manner that the likelihood of loosening of the implant is reduced, in contrast to the state of the art.

### Solution of the Task

The core idea of the solution of the task is to configure the shaft in a manner similar to that of a dowel known from the other sector. For this reason, the solution provides that the shaft of a joint part prosthesis, particularly for a finger joint, is configured at least in part as an expandable joint.

### Advantages of the Invention

One of the significant advantages of the invention is that because of the configuration of the joint part prosthesis according to the invention, it is no longer necessary to clear out the medullary space of the bone into which the implant is to be placed by means of working on it by hitting or hammering. Nevertheless, the joint part prosthesis can be fixed in place in simple and efficient manner. Since the shaft of the joint part prosthesis has rotation symmetry, it is sufficient to clear out the medullary space of the bone by means of rotational movements. In this way, damage to adjacent joints, in particular, caused by the hammering process required for clearing, is avoided.

Another advantage of the invention is that the joint part prosthesis can be inserted into the medullary space of a bone, with its shaft, without exerting great force, and in a further step can also be fixed in place there. The expansion sleeve that is provided and extends over at least part of the shaft is activated by means of an expansion body provided within the expansion sleeve, which can be displaced by the longitudinal displacement of the shaft. In an exemplary embodiment, it is advantageous if the expansion body is already an integral component of the expansion sleeve, so that by means of an additional tool, which preferably can be introduced by way of the proximal or distal component of the joint part prosthesis, respectively, can be moved in the direction of the component in question.

For this purpose, it is provided to configure the expansion body in such a manner that it is configured to be larger in diameter on its side that faces away from the component than in the remaining part of the shaft, so that the expansion sleeves are expanded away from one another by means of turning the expansion body in or out, for example using a screwdriver, and support themselves on the wall of the medullary space, or anchor themselves there, respectively.

In order for expansion in this form to be possible at all, it is advantageously provided that the expansion sleeve is configured in such a manner that a conventional shaft is configured in cannulated manner, and that corresponding slits are provided at its free end, which slits are also oriented in the longitudinal expanse of the shaft. The expansion body itself is mounted within the shaft, in the cannulated, bore-like region, and can also be turned clockwise and counterclockwise, preferably by way of a thread disposed within the shaft. Can be turned in one direction means that expansion is carried out, while in the other direction, expansion is cancelled out.

In a particularly preferred, advantageous embodiment, it is provided to configure turning tool and expansion body as a one-piece part, at first. The joint part prosthesis is structured in such a manner that a turning tool extends out of the component, which tool is at first connected, in one piece, with the expansion body. By means of turning the expansion body, the corresponding expansion of the shaft or of the expansion sleeve, respectively, is exerted, and when a certain torque is reached, the connection between the expansion sleeve and the turning tool breaks off; since a corresponding planned breakage point is provided at a defined location. The material properties are selected in such a manner that formation of chips is prevented.

Another advantageous embodiment provides that the expansion body extends, at least slightly, out of the cannulated region of the free end of the shaft, and thus covers the open region of the joint part implant. In this way, it is advantageously prevented that during insertion into the medullary space of the bone, additional marrow is entrained and destroyed, for example by means of the slits that are open toward the free end. Furthermore, the free end of the expansion body offers the advantage of functioning as an X-ray marker. In this way, it is possible for the precise position of the joint part prosthesis, which consists of plastic, for example, to be checked by way of an X-ray, to determine that it is correct.

A particularly preferred embodiment provides that anti-twist devices are provided on the outer wall of the shaft. The anti-twist devices are fin-like constructions that are disposed on the circumference at least once, and extend in the longitudinal expanse of the shaft. They are configured in such a manner that accordingly, they do not hinder the expansion process of the expansion sleeve, and additionally support anchoring of the implant within the medullary space.

A preferred embodiment of this anti-twist security device provides that it/they increase in their reaches, proceeding from the free end, in the direction of the distal or proximal component, so that twisting is still possible at insertion of the implant, and the farther the implant is introduced into the medullary space, the more twisting is restricted, accordingly.

In order to be able to install such anti-twist devices together with the shaft of a component, it is provided to clear the cavity accordingly, with rotation symmetry, and, as described above, using a clearing tool, by means of rotational and not hammering movements. An additional auxiliary means such as a gauge also supports clearing for the fin-like constructions, so that the implant can be inserted with precise fit.

In one exemplary embodiment, the joint part prosthesis consists of a material that is known from the state of the art.

Alternatives provide that a plastic can be used, particularly in order to achieve the modulus of elasticity similar to bone.

This also brings with it the particular advantage that the joint part prostheses can be produced by means of injection-molding methods.

Other advantageous embodiments are evident from the following description, the drawings, as well as the claims.

### DRAWINGS

The drawings show:

FIG. 1 a human hand, to illustrate the placement of the joint part prostheses;

FIG. 2 a first perspective view of the joint part prosthesis according to the invention, with a proximal component;

FIG. 3 another view of a perspective view of the joint part prosthesis according to the invention, with a distal component;

FIG. 4 a first section through the joint part prosthesis according to FIG. 2, extending through the plane IV;

FIG. 5 a section through the joint part prosthesis according to FIG. 3, extending through the plane V;

FIG. 6 a side view of the expansion screw disposed in the joint part prosthesis;

FIG. 7 a perspective view of the joint part prosthesis, but in the expanded state as compared with FIG. 2 and FIG. 3;

FIG. 8 another exemplary embodiment of the device according to the invention, with a first embodiment of an anti-twist security device;

FIG. 9 a third exemplary embodiment of the device according to the invention, with another embodiment of an anti-twist security device.

### DESCRIPTION OF EXEMPLARY EMBODIMENTS

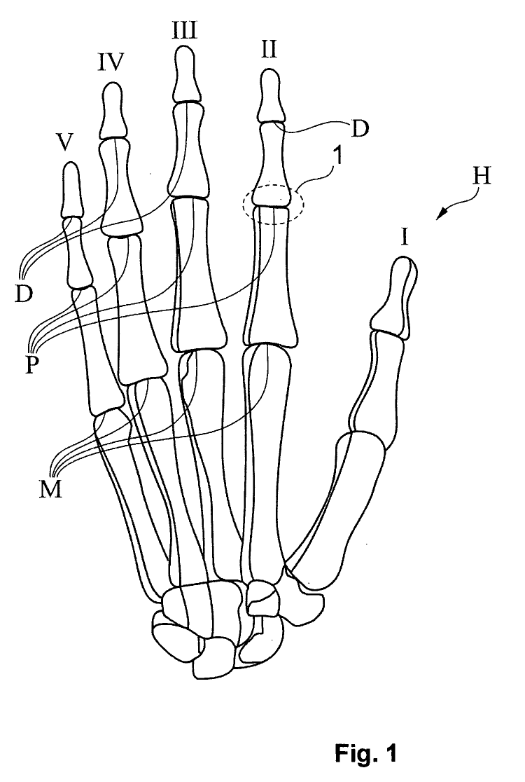

In FIG. 1, a hand H of a human body is shown in its bone structure. In order to define the nomenclature for affixing the joint part prostheses 1, as they are described below, the following designations are indicated: In FIG. 1, the distal interphalangeal joints D are disposed in the region of the fingertips. They are followed by the proximal interphalangeal joints P, and by the metacarporal phalangeal joints M disposed in the metacarpal region. The individual fingers are indicated with I to V.

Proximal Joint Part Prosthesis:

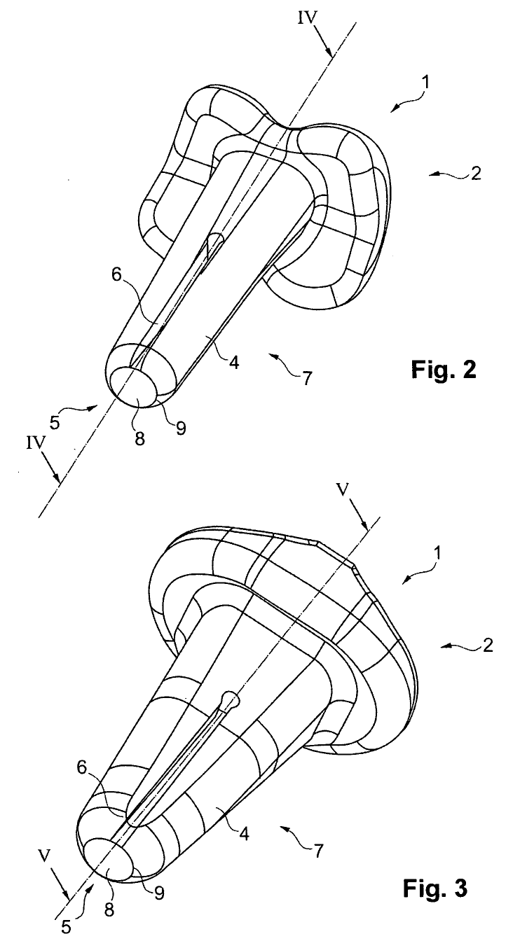

The joint part prostheses 1 described in the following can particularly be used in the distal as well as the proximal interphalangeal regions D, P, but also in the metacarporal phalangeal joints M. They are independent of the finger selection I to V. They merely differ in size and in the design of the proximal or distal components 2, 3, in each instance (FIG. 2, FIG. 3).

In FIG. 2, a perspective view of a joint part prosthesis 1 with a proximal component 2 is shown. The proximal component 2 has a slide surface, not shown in any detail in this embodiment, on which the distal component 3 shown in FIG. 3 can slide.

A shaft 4 extends to the rear from the proximal component 2. The shaft 4 extends almost perpendicular away from the proximal component 2, and has a narrowing toward its free end 5 in the exemplary embodiment shown here. It is configured to be round in cross-section.

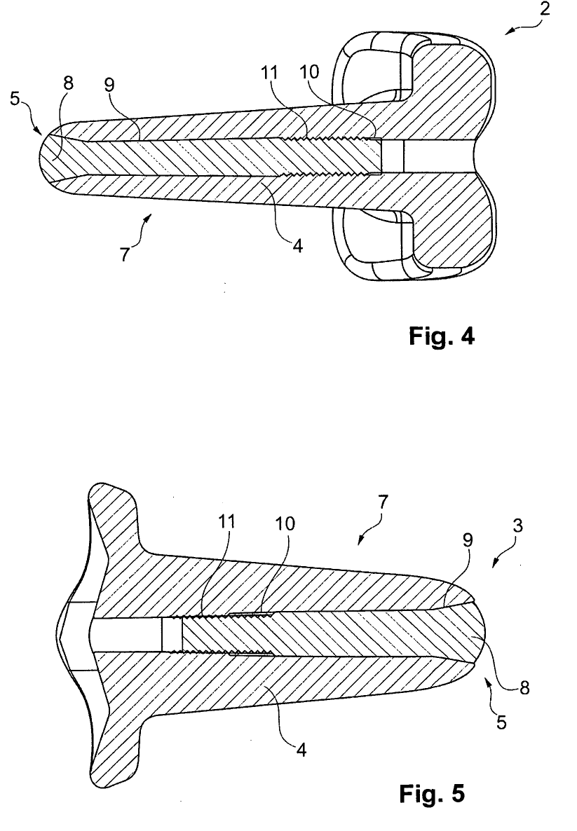

The free end 5 of the shaft 4 furthermore has slits 6. These slits 6 form an expansion sleeve 7 that is configured as at least part of the shaft 4. An expansion body 8 is disposed within the expansion sleeve 7. The expansion body 8 lies in a bore 9 provided within the expansion sleeve 7. As shown in FIG. 4, the bore 9 extends from the free end 5 of the shaft 4 all the way beyond the proximal component 2 of the joint part prosthesis 1. The bore 9 is configured to be wider at the free end 5 of the shaft 4 in comparison with the remainder of the shaft 4.

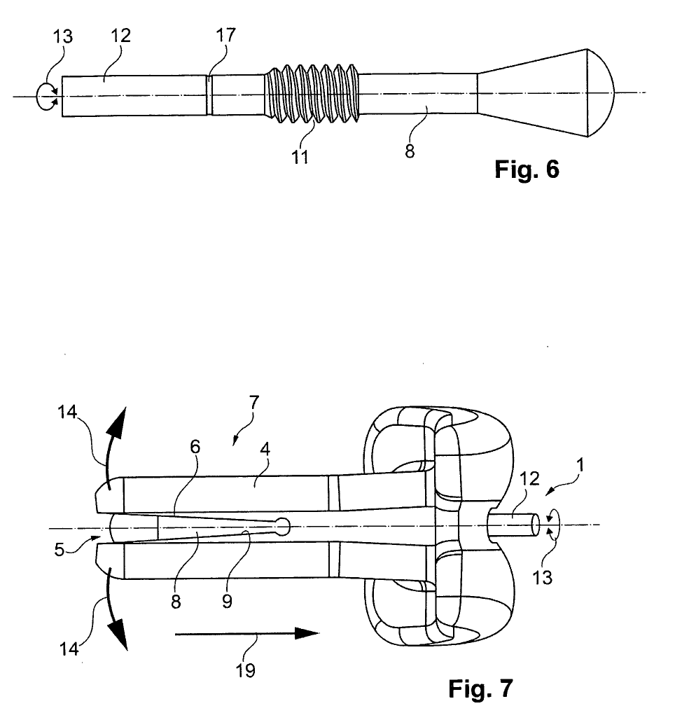

The bore 9 serves to accommodate the corresponding expansion body 8, as shown in FIG. 6. The free end 5 of the expansion body 8 is also configured to be wider, so that the expansion sleeve 7 is expanded in the event of movements of the expansion body 8 away from the free end 5, in the direction of the proximal component 2.

A particular embodiment of the expansion body 8 provides that this body projects away slightly beyond the free end 5. As a result, during introduction of the shaft 4 into a medullary space, the remaining marrow is protected, since the sharp edges of the slits 6 are prevented from injuring any marrow of the bone.

Furthermore, the wall of the shaft 4 is configured to be round, particularly in the region of the free end 5.

Distal Joint Part Prosthesis:

In FIG. 3, a perspective view of a joint part prosthesis 1 with a distal component 3 is shown. The distal component 3 has a slide surface, not shown in any detail in this embodiment, on which the proximal component 2 shown in FIG. 2 can slide.

A shaft 4 extends to the rear from the distal component 3. The shaft 4 extends almost perpendicular away from the distal component 3, and has a narrowing toward its free end 5 in the exemplary embodiment shown here. It is configured to be round in cross-section, so that adaptation to the medullary space of the bone is possible.

The free end 5 of the shaft 4 furthermore has slits 6. These slits 6 form an expansion sleeve 7 that is configured as at least part of the shaft 4. An expansion body 8 is disposed within the expansion sleeve 7. The expansion body 8 lies in a bore 9 provided within the expansion sleeve 7. As shown in FIG. 5, the bore 9 extends from the free end 5 of the shaft 4 all the way beyond the distal component 3 of the joint part prosthesis 1. The bore 9 is configured to be wider at the free end 5 of the shaft 4.

The bore 9 serves to accommodate the corresponding expansion body 8, as shown in FIG. 6. The free end 5 of the expansion body 8 is also configured to be wider, so that the expansion sleeve 7 is expanded in the event of movements of the expansion body 8 away from the free end 5, in the direction of the distal component 3.

A particular embodiment of the expansion body 8 provides that this body projects away slightly beyond the free end 5. As a result, during introduction of the shaft 4 into a medullary space, the remaining marrow is protected, since the sharp edges of the slits 6 are prevented from injuring any marrow of the bone.

Furthermore, the wall of the shaft 4 is configured to be round, particularly in the region of the free end 5. In this way, unintended injuries of the tissue are prevented from occurring when the joint part prosthesis 1 is pushed in.

Within the bore 9, a thread 10 is provided in the region of the shaft 4, which thread interacts with the expansion body 8 shown in FIG. 6. The expansion body 8 is introduced into the bore 9 and also has a thread 11, at least in the starting region, whereby the thread 11 additionally (along with the interaction with the thread 10 within the bore 9) cuts into expansion body 8, which preferably consists of plastic, by means of turning of the expansion body 8. In this way, it is guaranteed that the expansion body 8 maintains its position due to static friction, and thus the expansion that was carried out after the expansion body 8 was turned in is also maintained. The expansion body 8, in the embodiment shown in FIG. 6, has a planned breakage point 17. An auxiliary means 12 extends away from the planned breakage point 17, in the direction opposite to the free end, and is provided for turning the expansion body 8 in this and in the opposite direction.

In FIG. 7, a joint part prosthesis 1 is shown in a perspective view. It its starting position, the region of the expansion sleeve 7 is not under stress. This means that the expansion sleeve 7 assumes the shape as shown in FIGS. 2 and 3. The expansion body 8 is in its starting position.

In order to now move the expansion body 8 in the arrow direction 18, in order to carry out expansion of the expansion sleeve 7, the auxiliary means 12 (also shown in FIG. 6) is provided on the expansion body 8, which means interacts with the expansion body 8. The interaction is configured in such a manner that the expansion body 8 is moved in the arrow direction 18 by means of turning in the arrow direction 13, in that it moves in the arrow direction 19 by way of the thread 10/11 (shown in FIGS. 4 to 6), and in part, the thread 10 also cuts into the joint part prosthesis 1. With increasing expansion (arrows 14), the torque to be applied to the auxiliary means 12 increases, so that at a defined value, the auxiliary means 12 separates from the remainder of the expansion body 8, preferably at the planned breakage point 17 described in FIG. 6. This auxiliary means 12 can then be removed, without any corresponding chips being formed in the region of the joint part prosthesis 1. The planned breakage takes place within the joint part prosthesis 1, so that the slide surfaces continue to retain their desired property. The joint part prosthesis 1 is now fixed in place in the medullary space, and is fixed in place at least in the arrow direction 19, within the medullary space, by means of expansion of the expansion sleeve 7.

As an alternative, it can also be provided that in place of the auxiliary means 12, a tool is introduced, which is coupled with the expansion body 8 by way of an hexagonal socket wrench, for example.

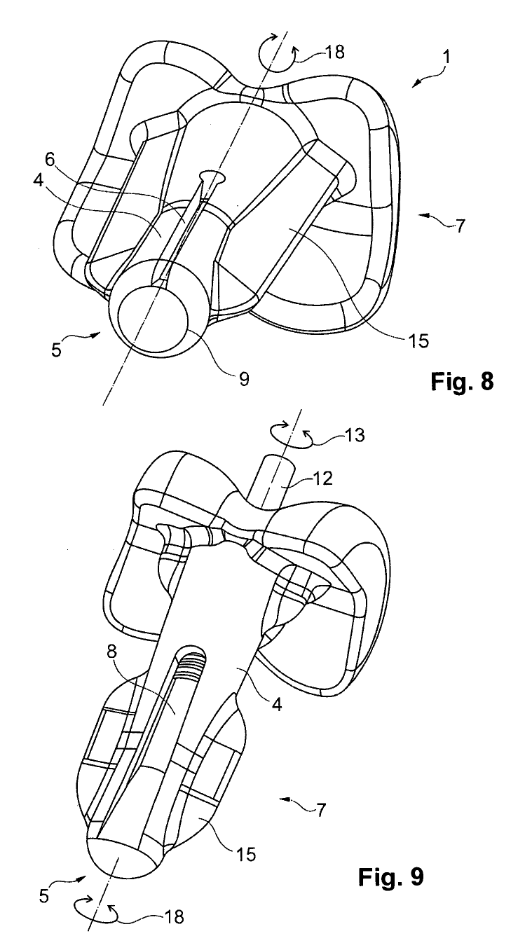

In FIG. 8, another exemplary embodiment of the embodiment of the invention according to the invention is shown. The joint part prosthesis 1 shown there has an anti-twist element 15 in addition to the properties already described previously. This anti-twist element 15 serves to fix a joint part prosthesis 1 that has been introduced into a medullary space, which space preferably takes up at least almost the same cross-section as the shaft configured together with the anti-twist element 15, in place in such a manner that when the expansion body 8 is turned in or counter to the arrow direction 18, turning of the joint part prosthesis 1 within the medullary space is no longer possible. The anti-twist element 15 provides fin-like configurations in the direction of the shaft 4, which extend along the shaft 4. Particularly in the region of the free end 5 of the shaft 4, it is provided to configure the anti-twist device 15 very slightly, so that during introduction, corresponding positioning within the medullary space is still possible. The deeper the joint part prosthesis 1 is introduced into the medullary space, the more the fin-like anti-twist devices 15, which is disposed on the circumference of the shaft 4 with at least a part, penetrate into the medullary space and press themselves into the marrow of the bone. A corresponding wedge effect occurs.

Another alternative embodiment of the joint part prosthesis 1 is shown in FIG. 9. This embodiment differs from the embodiment according to FIG. 8 in that the fin-like configuration of the anti-twist element 15 extends only partly over the longitudinal expanse of the shaft 4.

The anti-twist element 15 prevents rotational movement of the joint part prosthesis 1 in or opposite to the arrow direction 18. It serves, at least slightly, to bring about fixation of the joint part prosthesis 1 in the longitudinal expanse.

The joint part prosthesis 1 therefore demonstrates the property that a dowel-like configuration of the shaft 4 is made available by configuring the shaft 4, at least in part, as an expansion sleeve 7, which configuration brings about the result that a joint part prosthesis 1 that is introduced into a medullary space of the bone is fixed in place in the introduction directions, or in the opposite direction, respectively. Loosening of the joint part prosthesis 1, brought about by mechanical movements, but also by a reduction in the marrow in the medullary space of the bone or by other chemical, biological, or physical properties, is circumvented. Furthermore, the shaft 4 is configured to be rough and has multiple openings into which the corresponding marrow can grow, so that a shape-fit and force-fit connection of the joint part prosthesis 1 with the remainder of the bone can be formed. The anti-twist element 15 supports orientation of the joint part prosthesis 1 in the correct position, and prevents twisting in or opposite to the arrow direction 18. By means of the joint part prosthesis 1 according to the invention, operative adaptation and positioning of a joint part prosthesis 1 that is particularly gentle on the joint becomes possible.

Reference Symbol List

| D | distal interphalangeal joints |
|---|---|
| P | proximal interphalangeal joints |
| M | metacarporal phalangeal joints |
| 1 | joint part prosthesis |
| 2 | proximal component |
| 3 | distal component |
| 4 | shaft |
| 5 | free end |
| 6 | slits |
| 7 | expansion sleeve |
| 8 | expansion body |
| 9 | bore |
| 10 | thread |
| 11 | arrow |
| 12 | auxiliary means |
| 13 | arrow direction |
| 14 | arrow direction |
| 15 | anti-twist device |
| 16 | arrow direction |
| 17 | planned breakage point |
| 18 | arrow direction |

1. Joint part prosthesis, particularly for a finger joint, consisting of either a proximal or a distal component, as well as a shaft that extends away from this component, to be mounted in a bone, wherein the shaft (4) is configured at least in part as an expansion sleeve (7).

2. Joint part prosthesis according to claim 1, wherein an expansion body (8) that can be displaced in the longitudinal expanse of the shaft (4) is provided within the expansion sleeve (7).

3. Joint part prosthesis according to claim 1, wherein the expansion sleeve (7) is formed by means of slits (6) provided at the free end (5) of the shaft (4).

4. Joint part prosthesis according to claim 1, wherein the expansion body (8) extends all the way to the free end (5) of the shaft (4).

5. Joint part prosthesis according to claim 1, wherein in the non-expanded state of the shaft (4), the free end (5) of the shaft (4) forms a closed unit, by means of the expansion body (8).

6. Joint part prosthesis according to claim 1, wherein the expansion body (8) consists of plastic.

7. Joint part prosthesis according to claim 1, wherein a bore (9) is provided within the shaft (4), for accommodating the expansion body (8), whereby a thread (10) is provided within the bore (9), which interacts with the expansion body (8).

8. Joint part prosthesis according to claim 1, wherein the bore (9) extends all the way into the region of the proximal component (2) or distal component (3), respectively.

9. Joint part prosthesis according to claim 8, wherein an auxiliary means (12) for moving the expansion body (8) in or opposite to the arrow direction (11) can be introduced into the bore (9).

10. Joint part prosthesis according to claim 1, wherein the expansion body (8) comprises an X-ray marker.

11. Joint part prosthesis according to claim 1, wherein the shaft (4) consists of plastic.

12. Joint part prosthesis according to claim 1, wherein the shaft (4) comprises a rough surface.

13. Joint part prosthesis according to claim 9, wherein the auxiliary means (12) and the expansion body (8) form a one-part piece.

14. Joint part prosthesis according to claim 13, wherein a planned breakage point is provided between the auxiliary means (12) and the expansion body (8).

15. Joint part prosthesis according to claim 14, wherein the planned breakage point acts in torque-limiting manner.
